(19) United States
(12) Patent Application Publication
(10) Pub. No.: US 2020/0392058 A1
(43) Pub. Date: Dec. 17, 2020

(54) METHOD FOR PREPARING 1,3-PROPANEDIOL BY COUPLING ETHYLENE OXIDE WITH SYNGAS

(71) Applicant: Xiamen University, Xiamen (CN)

(72) Inventors: Hongping Zhu, Xiamen (CN); Jinbo Zhao, Xiamen (CN); Yunbao Jiang, Xiamen (CN); Enyi Lai, Xiamen (CN); Wenjun Jiang, Xiamen (CN); Qiaozhu Jiang, Xiamen (CN)

(21) Appl. No.: 16/827,845

(22) Filed: Mar. 24, 2020

(30) Foreign Application Priority Data
Jun. 13, 2019 (CN) 201910509398.4

(51) Int. Cl. C07C 29/34; B01J 31/22; B01J 31/20

(52) U.S. Cl. CPC C07C 29/34; B01J 31/2217; B01J 2231/648; B01J 2531/845; B01J 2531/004; B01J 31/20

(57) ABSTRACT

A catalytic reaction of ethylene oxide (EO) coupling with syngas to produce 1,3-propanediol (1,3-PDO) is disclosed. The catalytic reaction of EO, carbon monoxide and the alcohol uses a N,O-ligand coordinated metal complex catalyst. The reaction is carried out in an organic solvent in the presence of an additive at the temperature of 30-190° C. and the CO pressure of 1-150 atm for 0.1-200 h to prepare 3-hydroxypropinate (3HP). The catalytic reaction of 3HP with dihydrogen uses a copper-containing mixed metal silicon oxide catalyst with a molecular formula of M′ᵤCuᵥSi_{y}O_{z}. The reaction is carried out at 80-400° C. and 20-150 atm for 0.1-200 h to prepare the 1,3-PDO. The yield of the 1,3-PDO can reach to 73%. The alcohol byproduct generated in the second step catalytic hydrogenation reaction can be recycled to use for the first step catalytic reaction by the ring opening-carbonylation-esterification.

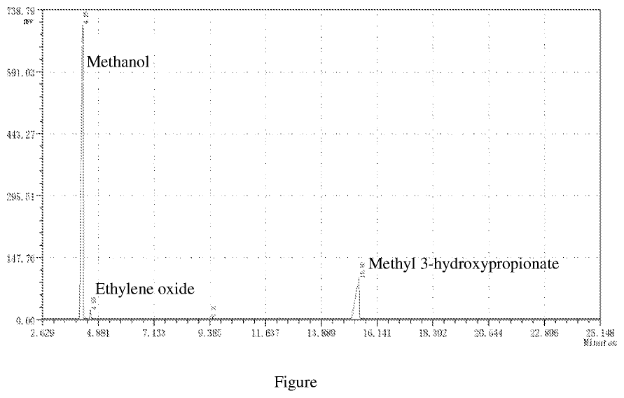

### TECHNICAL FIELD

This invention relates to a method for preparing 1,3-propanediol (1,3-PDO) by coupling ethylene oxide (EO) with syngas, and in particular, to a two-step catalytic reaction method. First, an alcohol molecule is introduced to react with EO and carbon monoxide through ring opening-carbonylation-esterification to produce 3-hydroxypropionate (3HP). Second, the obtained 3HP is subjected to a hydrogenation reaction to produce a target 1,3-PDO product and an alcohol molecule byproduct. Particularly, the obtained alcohol byproduct is recycled to the first reaction. The successful implementation of the present invention is closely related to the two-step reaction process by the use of separated, differed catalyst in each reaction.

### BACKGROUND

1,3-propanediol (1,3-PDO) is an antifreeze and a synthetic raw material for a variety of plasticizers, detergents, preservatives and emulsifiers. A more important use of 1,3-PDO is as a monomer for polymerization. Polymerization of 1,3-PDO with terephthalic acid produces polytrimethylene terephthalate (PTT). PTT is a polyester material that can be used as fiber for manufacturing high-quality carpets and other fabrics, and can also be used for plastic. In this field, an important target is to invent a 1,3-PDO preparation route which is cost-effective and technologically advanced.

As early as 1960, Eisenmann, Yamartino, and Howard Jr. in the UK studied the reaction of propylene oxide (PO), CO and methanol under the catalysis of Co₂(CO)₈. The reaction generated a methyl 3-hydroxybutyrate primary product with a yield of 20.8-40.3%, accompanied by other byproducts (J. Org. Chem. 1961, 26, 2102-2104). The reaction conditions were relatively harsh, with the CO pressure of 53-400 bars and the temperature of 110-190° C. In 1963, Heck, who worked at the research center of Hercules Powder in the United States, reported the use of NaCo(CO)₄ to catalyze the reaction of ethylene oxide (EO), CO and methanol. The reaction generated methyl 3-hydroxypropionate (3-HMP) with a yield of 55% at the CO pressure of 133 atm and the temperature of 65° C., but there were too many byproducts with complicated compositions (J. Am. Chem. Soc. 1963, 85, 1460-1463). In 1996, the Shell company in the United States announced the industrial production of 1,3-PDO. It had disclosed that the use of EO, CO, and H₂ as raw materials to prepare 1,3-PDO was by a one-step hydroformylation method (U.S. Pat. No. 3,463,819). The Shell company also disclosed a two-step method, that is, EO, CO and H₂ were first hydroformylated to produce 3-hydroxypropanal, and then the 3-hydroxypropanal was hydrogenated to obtain 1,3-PDO (U.S. Pat. No. 5,545,766).

In addition to the hydroformylation method announced by the Shell Company, other methods had also been reported. Of these methods, a prominent method was based on the reaction reported by Heck in 1963. This method was called hydromethylesterification method so as to distinguish from the Shell's hydroformylation method. In the hydromethylesterification field, in 1990 and 1992, Eastman Kodak disclosed a catalyst system composed of rhodium, ruthenium, phosphine ligand and Group 5 promoter (U.S. Pat. Nos. 4,973,741 and 5,135,901). The catalyst system catalyzed the reaction of EO, CO, H₂ and C₁₋₆ linear alcohol or benzyl alcohol to prepare 3-hydroxypropionate (3HP). In an example, at the reaction temperature of 70° C. and the initial pressure of 137.9 bars, the EO had a conversion rate of 26.3-90.3% and the 3-HMP had a selectivity of 12.3-43.5%. In 2002 and 2003, Samsung Electronics disclosed a catalyst system composed of cobalt and nitrogen ligand to catalyze the reaction of EO, CO and alcohol to prepare 3HP (U.S. Pat. No. 6,348,632B1 and U.S. Pat. No. 6,521,801B1). In an example, at the reaction temperature of 60-80° C. and the initial pressure of 34-80 bars, the EO had a conversion rate of 45.19-95.27% and the 3-HMP had a selectivity of 78.45-93.31%. In 2002, Samsung Electronics disclosed a method for preparing 1,3-PDO by the hydrogenation of 3HP (CN1355160A). This method used catalytic Cu—Si—O and a small amount of auxiliaries such as Re, Pd, Ru, Pt, Rh, Ag, Se, Te, Mo and Mn (0.001-10 mol % based on copper). In an example, at the reaction temperature of 145-150° C. and the initial pressure of 62-69 bars, the 3-HMP had a conversion rate of 82.90-97.92% and the 1,3-PDO had a selectivity of 84.28-89.63%.

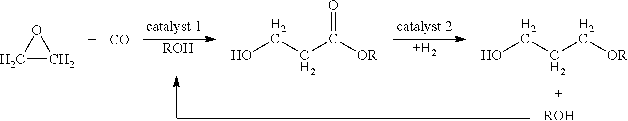

Scheme 1 A general chemical process for preparing 1,3-PDO by coupling EO with syngas

The key to the industrialization of the hydromethylesterification method, is to design a reasonable catalyst system and a 1,3-PDO preparation route which is cost-effective and technologically advanced. The recent laboratory research has shown that the two steps can be coupled. In the first step, EO, CO and alcohol are subject to ring-opening-carbonylation-esterification to generate 3HP. In the second step, the generated 3HP is hydrogenated to generate a 1,3-PDO product and an alcohol byproduct. The alcohol byproduct in the second step is recycled for the first step reaction, as shown in scheme 1. Different from the Shell's two-step hydroformylation method, this method introduces an alcohol into the EO and syngas to produce the 3HP intermediate.

The method uses different catalyst systems in the first and second steps so as to improve the efficiency. In the first step, a N,O-ligand stabilized metal catalyst efficiently catalyzes the reaction under mild conditions. The N,O ligand is a non-phosphine ligand, which is different from the above-mentioned nitrogen-containing ligand (U.S. Pat. No. 6,348,632B1, U.S. Pat. No. 6,521,801B1, CN106431921A, CN107349962A and CN107459451A). The conversion rate of the EO reaches 99% and the 3-HP selectivity reaches 98%. In the second step, a copper-containing mixed metal silicon oxide catalyst effectively catalyzes the hydrogenation reaction. The yield of the 1,3-PDO reaches 73% and the alcohol byproduct is separated for use in the first step.

### SUMMARY

In view of the above, the present invention particularly provides a N,O-ligand coordinated metal complex catalyst, and a structure thereof is shown in the following formula:

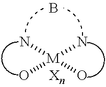

where in the above structure, a metal M represents one of nickel, cobalt, ruthenium, rhodium, palladium, platinum, osmium, iridium, iron, copper and chromium, preferably one of cobalt, ruthenium, rhodium and iridium; a N,O ligand represents an organic group with N and O as coordinating atoms; N and O both have a σ-bond interaction with the metal M in the center; B represents a bridging organic group connecting two N,O ligands and bonded to a nitrogen atom in the two ligands; X represents an anionic group or an atom or a neutral group; n represents a number of X; n is able to form a reliable molecule where the coordination number of the central M is reasonably maintained after the coordination of the N,O ligand at the M; X is preferably one selected from H, CO, halogen, pseudohalogen, alkyl, alkoxyl, alkyl sulfydryl, aryl, benzyl, amino, hydroxyl and carboxylic group; in particular, X is one selected from H, CO, Cl, SCN, methyl, ethyl, methoxyl, ethoxyl, isopropoxyl, phenyl, benzyl, methylamino, ethylamino, OH, formyl and acetoxyl.

Based on the N,O-ligand coordinated metal complex catalyst, ethylene oxide (EO), carbon monoxide and an alcohol molecule react in an organic solvent with an additive or additives under certain temperature and pressure in a controlled time. The additive is one selected from a basic metal oxide, a main group metal alkoxyl compound, a main group metal amino compound, a main group metal carboxyl compound, a metal carbonyl compound and a Lewis basic nitrogen-containing compound. In particular, the basic metal oxide is one selected from lithium oxide, sodium oxide, potassium oxide and magnesium oxide; the main group metal alkoxy compound is one selected from lithium alkoxide, sodium alkoxide, potassium alkoxide, magnesium alkoxide, calcium alkoxide, strontium alkoxide, barium alkoxide, aluminum alkoxide and gallium alkoxide, and is preferably one selected from lithium methoxide, sodium methoxide, potassium methoxide, lithium ethoxide, sodium ethoxide, potassium ethoxide, lithium isopropoxide, sodium isopropoxide, potassium isopropoxide, magnesium methoxide, magnesium ethoxide and magnesium isopropoxide; the main group metal amino compound is one selected from lithium alkylamino, sodium alkylamino, potassium alkylamino, magnesium alkylamino and calcium alkylamino, and is preferably one selected from lithium methylamino, sodium methylamino, potassium methylamino, magnesium methylamino, calcium methylamino, lithium ethylamino, sodium ethylamino, potassium ethylamino, magnesium ethylamino, calcium ethylamino, lithium trimethylsilylamino, sodium trimethylsilylamino and potassium trimethylsilylamino; the main group metal carboxyl compound is one selected from lithium formate, sodium formate, potassium formate, lithium acetate, sodium acetate, potassium acetate, magnesium formate, magnesium acetate, calcium formate and calcium acetate; the metal carbonyl compound is one selected from cobalt carbonyl, iron carbonyl, molybdenum carbonyl, tungsten carbonyl, chromium carbonyl, ruthenium carbonyl, iridium carbonyl and rhodium carbonyl; the Lewis basic nitrogen-containing compound is one selected from alkylamine and a nitrogen-containing heterocyclic compound, and is preferably one selected from trimethylamine, triethylamine, triisopropylamine, tri-tert-butylamine, dimethylphenylamine, pyrrole, pyrazol, triazole, imidazole, indole, pyridine, 2-hydroxypyridine, 3-hydroxypyridine and 4-hydroxypyridine.

In the above reaction, the alcohol molecule is one selected from a C₁-C₂₀ alcohol, and is preferably one selected from methanol, ethanol, propanol, butanol and pentanol. The organic solvent is one selected from alcohol, ether, saturated alkane and saturated aromatic hydrocarbon, and is preferably one selected from methanol, ethanol, propanol, butanol, pentanol, dimethyl ether, diethyl ether, methyl tert-butyl ether, tetrahydrofuran, 1,4-dioxane, pentane, hexane, heptane, octane, benzene, methylbenzene, dimethylbenzene, trimethylbenzene.

The above reaction is carried out at the temperature of 30-190° C. and the CO pressure of 1-150 atm for 0.1-200 h. The reaction temperature may be preferably 30-150° C., the reaction pressure may be preferably 1-120 atm, and the reaction time may be preferably 1-180 h.

In order to further use a 3-hydroxypropionate (3HP) product from the above reaction for a subsequent hydrogenation reaction, a reaction product system needs to undergo phase separation. The present invention provides a phase separation method to form an aqueous phase, an organic phase and a precipitate phase, and to allow the 3HP product to maximally remain in the organic phase.

The method provided by the present invention is to add distilled water to the reaction product system to form the aqueous phase, add an organic solvent to form the organic phase, and add a precipitating agent to generate the precipitate phase. The organic solvent is one selected from alcohol, ether, saturated alkane, saturated aromatic hydrocarbon, ester, C₅ and above long-chain olefin and C₄ and above long-chain alkyne, and is preferably one selected from diethyl ether, methyl tert-butyl ether, pentane, hexane, heptane, benzene, methylbenzene, hexene and octene; the precipitating agent is one selected from Bronsted acid, Bronsted base, Lewis acid, Lewis base, silica gel, molecular sieve, alumina, kaolin, hydrotalcite and ion exchange resin, and is preferably one selected from NaOH, KOH, Na₂CO₃, NaHCO₃, K₂CO₃, KHCO₃, silica gel, molecular sieve, alumina, kaolin, hydrotalcite and ion exchange resin; the phase separation method is one selected from extraction, standing, centrifuge separation, filtration, distillation, column chromatography separation and vacuum extraction.

In the organic phase generated by the phase separation above, the 3HP product and the organic solvent may be separated or not. To separate the 3HP product from the organic solvent, a separation method is one selected from distillation, rectification, column chromatography separation and vacuum extraction.

The present invention also provides a catalytic hydrogenation reaction method for the 3HP. This method uses a copper-containing mixed metal silicon oxide catalyst, and in particular, a copper-containing mixed metal silicon oxide having a general formula of M′ᵤCuᵥSi_{y}O_{z}. In the formula, u, v, y and z maintain a zero overall oxidation number of molecules. M′ is one or two or three of zinc, manganese, barium, lanthanide series metal, cobalt, silver, gold, nickel and potassium. Preferably, the copper-containing mixed metal silicon oxide includes 20-70% copper, 10-30% silicon, 10-40% oxygen and 0.5-10% M′.

When the 3HP-containing organic phase obtained by the phase separation above is used for a catalytic hydrogenation reaction, the reaction does not require a solvent. When the 3HP obtained by the separation above is used for a catalytic hydrogenation reaction, the reaction may or may not require an organic solvent, which is one selected from ether, saturated alkane and saturated aromatic hydrocarbon, and preferably selected from pentane, hexane, heptane, benzene and methylbenzene.

The catalytic hydrogenation reaction of the 3HP is carried out at the temperature of 80-400° C. and the H₂ pressure of 20-150 atm for 0.1-200 h. The reaction temperature may be preferably 90-270° C., the H₂ pressure may be preferably 30-120 atm, and the reaction time may be preferably 0.5-150 h.

A product system of the hydrogenation reaction mainly includes a 1,3-propanediol (1,3-PDO) primary product and an alcohol byproduct. The 1,3-PDO primary product and the alcohol byproduct are separated by using a method selected from distillation, rectification, column chromatography separation and vacuum extraction. The alcohol byproduct is separated and recycled for a catalytic reaction with EO and carbon monoxide to prepare the 3HP.

### Additional Summary

The present invention uses a hydromethylesterification method to couple ethylene oxide (EO) with syngas to prepare 1,3-propanediol (1,3-PDO). The first reaction step of the EO, carbon monoxide and an alcohol to produce 3-hydroxypropionate (3HP) is sequenced with the second reaction step by the 3HP and hydrogen to give the 1,3-PDO and the alcohol. The alcohol molecule is introduced into the first step reaction to produce the 3HP intermediate, and the 3HP intermediate is subject to the second step hydrogenation to regenerate the alcohol molecule. By introducing the alcohol molecule, this method achieves the atom economy of the reaction of the EO and the syngas to prepare the 1,3-PDO. This hydromethylesterification method is different from a hydroformylation method disclosed by the Shell company, which prepares 1,3-PDO by using EO, carbon monoxide and dihydrogen.

In the hydromethylesterification method, the two-step catalytic reaction uses different catalysts with different catalytic reaction mechanisms. The first step reaction is a ring-opening-carbonylation-esterification reaction, including ring opening of the EO, insertion of the carbon monoxide and esterification with the alcohol. The second step reaction is a double hydrogenation reaction, that is, the 3HP reacts with a dihydrogen molecule (H₂) to yield 3-hydroxypropanal and the alcohol, and the 3-hydroxypropanal further reacts with a dihydrogen molecule (H₂) to produce the 1,3-PDO.

The first step reaction uses a N,O-ligand coordinated metal complex catalyst and the second step reaction uses a copper-containing mixed metal silicon oxide catalyst having a general formula of M′ᵤCuᵥSi_{y}O_{z}.

The catalytic reaction of the EO, the carbon monoxide and the alcohol is carried out in an organic solvent including an alcohol. The alcohol serves as not only the organic solvent but also the reactant. In this regard, the alcohol is excessed in amount, as greatly promotes the kinetic conversion of the reaction for yielding the 3HP product.

In the present invention, the catalytic reaction of the EO, the carbon monoxide and the alcohol in the organic solvent can be applied in a batch reaction technique process, a continuous reaction technique process or a combination of these two processes thereof. The catalytic reaction of the 3HP and the dihydrogen can be conducted in a batch reaction technique process, a continuous reaction technique process or a combination of these two processes thereof. The two catalytic reactions are sequenced, so that the alcohol produced from the reaction of the 3HP with the dihydrogen can be separated and recycled for the reaction of the EO, the carbon monoxide and the alcohol in the organic solvent.

### BRIEF DESCRIPTION OF THE DRAWINGS

FIGURE shows a gas chromatographic spectrum of product yielded in Example 9.

### DETAILED DESCRIPTION

The present invention is described in detail below for practice. Those skilled in the art should understand that the present invention is not limited to the following content disclosed.

The catalytic reaction of ethylene oxide (EO), carbon monoxide and alcohol in organic solvent was generally operated as follows.

To a 2000 mL reactor, flushed with N₂ for at least three times, was added a N,O-ligand coordinated metal complex, an additive, EO, an alcohol and an organic solvent in sequence, where the catalyst has a concentration of 1 mmol/L, the additive has a concentration of 2 mmol/L, the EO has a concentration of 400 mmol/L, and the alcohol has a concentration of 400 mmol/L. The reactor was closed, and the temperature, the stirring rate and the reaction time were set. After reaching to the settled temperature, the carbon monoxide was switched on to the reactor with the settled pressure to start the reaction. By the settled reaction time, the carbon monoxide was switched off. The reactor was cooled to 0° C., and then the excess unreacted carbon monoxide was slowly released through the vent valve collected to the reactor. The reaction solution was poured out for treatment.

The quantitative and qualitative analysis of the reaction products was analyzed by means of the GC and GC-MS spectra. The reaction sample for analysis obtained by Gas chromatographic analyses of the product mixture were made on a gas chromatograph. The analysis data and the calculation data thereof were recorded in Table 1.

The treatment process for separation was described as follows.

To the solution was added a double volume of H₂O and then an equivalent volume of CH₂Cl₂. A two-phase mixture solution was formed, which was subjected to the separation by using the separating funnel. The organic component was collected and dried with Na₂SO₄. The rectification allowed separation of 3-HP, methanol, and CH₂Cl₂, respectively, as the main component.

### Examples 1 to 19

These examples select a catalyst with a structure shown in the following.

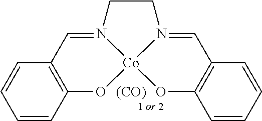

In order to fully evaluate the reactivity of this catalyst in combining the sodium methoxide as the additive in methanol solvent, the experiments were carried out by adopting varied conditions. First, the reactions were carried out at the CO pressure of 50 atm within 4 h by varying the temperatures from 40 to 60, 80, 100, 120 and finally 140° C., and the results are recorded in Table 1 (Examples 1 to 6). Second, the reactions were carried out at the temperature of 80° C. within 4 h by varying the CO pressure from 5 to 10, 30, 50, 70, 90, 100 and finally 120 atm, and the results are recorded in Examples 7 to 13 in Table 1. Third, the reactions were carried out at the temperature of 80° C. and the CO pressure of 30 atm by varying the reaction time from 1 to 2, 3, 5 and 6 h, respectively, and the results are recorded in Examples 14 to 18 in Table 1.

Table 1 Summary of the reaction results

| | | | | | Selectivity |
|---|---|---|---|---|---|
| | | | Reaction | EO | of |
| | Temperature | Pressure | time | Conversion | 3-HMP |
| Example | (° C.) | (atm) | (h) | Rate (%) | (%) |
| 1 | 40 | 50 | 4 | 22.3 | 84.3 |
| 2 | 60 | 50 | 4 | 59.0 | 86.2 |
| 3 | 80 | 50 | 4 | 96.9 | 96.5 |
| 4 | 100 | 50 | 4 | 95.4 | 69.5 |
| 5 | 120 | 50 | 4 | 96.4 | 56.7 |
| 6 | 140 | 50 | 4 | 98.3 | 26.8 |
| 7 | 80 | 5 | 4 | 23.4 | 76.8 |
| 8 | 80 | 10 | 4 | 66.7 | 80.1 |
| 9 | 80 | 30 | 4 | 96.3 | 95.5 |
| 10 | 80 | 70 | 4 | 94.5 | 78.2 |
| 11 | 80 | 90 | 4 | 96.2 | 96.1 |
| 12 | 80 | 100 | 4 | 97.8 | 97.3 |
| 13 | 80 | 120 | 4 | 98.9 | 98.8 |
| 14 | 80 | 30 | 1 | 26.3 | 86.5 |
| 15 | 80 | 30 | 2 | 43.8 | 89.9 |
| 16 | 80 | 30 | 3 | 56.2 | 87.1 |
| 17 | 80 | 30 | 5 | 98.8 | 95.2 |
| 18 | 80 | 30 | 6 | 99.6 | 98.6 |

### Examples 19 to 48

By using the catalyst shown in the above, reactions were carried out in methanol as a solvent in the presence of different additives by setting the temperature at 80° C., the CO pressure of 30 atm, and the reaction time for 4 h. The results obtained are recorded in Examples 19 to 32 of Table 2.

By using the catalyst shown in the above, reactions were carried out in the presence of pyridine as an additive in different solvents by setting the temperature at 80° C., the CO pressure of 30 atm, and the reaction time for 4 h, where methanol was used as a reactant with the concentration of 400 mmol/L. The results are recorded in Examples 33 to 41 of Table 2.

By using the catalyst shown in the above and introducing different alcohols, reactions were carried out in methyl tert-butyl ether as a solvent in the presence of pyridine as an additive at the temperature of 80° C. and the CO pressure of 30 atm within 4 h. The results are recorded in Examples 42 to 48 of Table 2

Table 2 Summary of the reaction results

| | | | | EO | Selectivity |
|---|---|---|---|---|---|
| | | | | Conversion | of 3HP |
| Example | Alcohol | Additive | Solvent | Rate (%) | (%) |
| 19 | — | Sodium ethoxide | Methanol | 56.8 | 78.9 |
| 20 | — | Magnesium ethoxide | Methanol | 46.5 | 86.4 |
| 21 | — | Lithium | Methanol | 52.3 | 88.6 |
| | | diisopropylamide | | | |
| 22 | — | Sodium acetate | Methanol | 31.1 | 91.2 |
| 23 | — | Sodium carbonate | Methanol | 48.5 | 52.6 |
| 24 | — | Triethylamine | Methanol | 47.9 | 90.1 |
| 25 | — | Lithium | Methanol | 56.2 | 76.2 |
| | | dimethylphenylamine | | | |
| 26 | — | Pyridine | Methanol | 68.9 | 93.5 |
| 27 | — | Pyrrole | Methanol | 70.2 | 91.6 |
| 28 | — | 2-hydroxypyridine | Methanol | 68.5 | 90.1 |
| 29 | — | 3-hydroxypyridine | Methanol | 96.2 | 98.6 |
| 30 | — | 4-hydroxypyridine | Methanol | 86.8 | 96.8 |
| 31 | — | Imidazole | Methanol | 78.5 | 90.2 |
| 32 | — | Indole | Methanol | 50.6 | 80.1 |
| 33 | Methanol | Pyridine | Diethyl ether | 86.5 | 90.2 |
| 34 | Methanol | Pyridine | Tetrahydrofuran | 90.1 | 88.2 |
| 35 | Methanol | Pyridine | Methyl | 80.6 | 84.9 |
| | | | tert-butyl ether | | |
| 36 | Methanol | Pyridine | Pentane | 85.3 | 90.2 |
| 37 | Methanol | Pyridine | Hexane | 86.2 | 87.6 |
| 38 | Methanol | Pyridine | Benzene | 86.9 | 67.5 |
| 39 | Methanol | Pyridine | Methylbenzene | 90.6 | 56.8 |
| 40 | Methanol | Pyridine | 1-hexene | 78.9 | 60.8 |
| 41 | Methanol | Pyridine | 1-octene | 70.6 | 68.1 |
| 42 | Ethanol | Pyridine | Methyl | 74.6 | 80.5 |
| | | | tert-butyl ether | | |
| 43 | N-propanol | Pyridine | Methyl | 86.2 | 86.9 |
| | | | tert-butyl ether | | |
| 44 | Isopropanol | Pyridine | Methyl | 47.5 | 43.6 |
| | | | tert-butyl ether | | |
| 45 | N-butanol | Pyridine | Methyl | 72.3 | 79.8 |
| | | | tert-butyl ether | | |
| 46 | Isobutanol | Pyridine | Methyl | 45.8 | 32.9 |
| | | | tert-butyl ether | | |
| 47 | Tert-butanol | Pyridine | Methyl | 42.1 | 43.0 |
| | | | tert-butyl ether | | |
| 48 | N-pentanol | Pyridine | Methyl | 86.7 | 90.7 |
| | | | tert-butyl ether | | |

### Examples 49 to 55

In this section, the examples are designed to study the reactivity of the different metal complex catalysts incorporated with the N,O-ligand shown in forming catalyst. Reactions were carried out by using methanol as a reactant, pyridine as an additive, and methyl tert-butyl ether as a solvent at the temperature of 80° C. and the CO pressure of 30 atm for 4 h. The results are recorded in Examples 49 to 32 of Table 3.

Table 3 Summary of the reaction results

| | | EO Conversion | Selectivity of |
|---|---|---|---|
| Example | Metal | Rate (%) | 3-HMP (%) |
| 49 | Ruthenium | 76.9 | 50.8 |
| 50 | Rhodium | 60.6 | 61.3 |
| 51 | Palladium | 33.5 | 43.4 |
| 52 | Platinum | 45.3 | 56.1 |
| 53 | Osmium | 30.6 | 39.7 |
| 54 | Iridium | 20.9 | 16.9 |
| 55 | Iron | 13.8 | 7.9 |

### Examples 49 to 55

In this section, the examples are designed to study the reactivity of different cobalt complex catalysts coordinated with different N,O-ligands. Reactions were carried out at the temperature of 80° C. and the CO pressure of 30 atm for 4 h by using methanol as a reactant, pyridine as an additive, methyl tert-butyl ether as a solvent. The results are recorded in Examples 49 to 55 of Table 4.

Catalyst II with a structure shown in the following is selected.

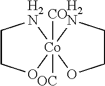

Catalyst III with a structure shown in the following is selected.

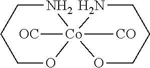

Catalyst IV with a structure shown in the following is selected.

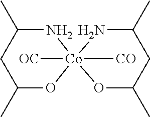

Catalyst V with a structure shown in the following is selected.

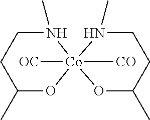

Catalyst VI with a structure shown in the following is selected.

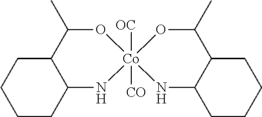

Catalyst VII with a structure shown in the following is selected.

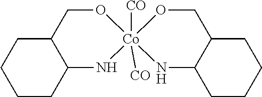

Catalyst VIII with a structure shown in the following is selected.

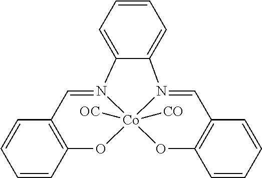

Catalyst IX with a structure shown in the following is selected.

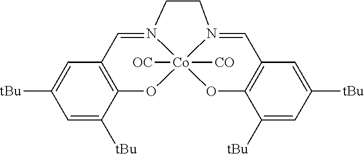

Catalyst X with a structure shown in the following is selected.

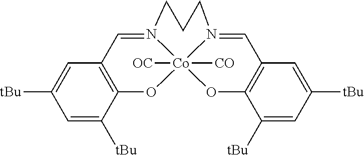

Catalyst XI with a structure shown in the following is selected.

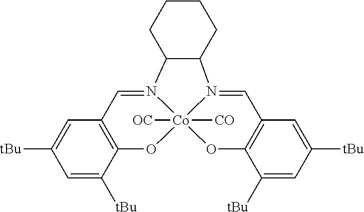

Catalyst XII with a structure shown in the following is selected.

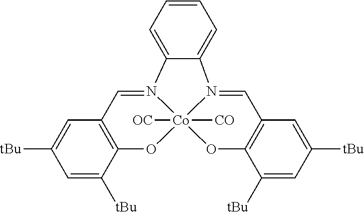

Catalyst XIII with a structure shown in the following is selected.

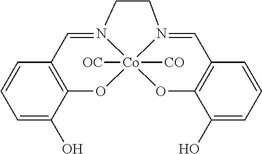

Catalyst XIV with a structure shown in the following is selected.

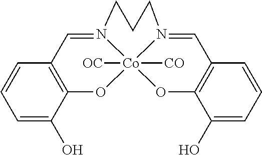

Catalyst XV with a structure shown in the following is selected.

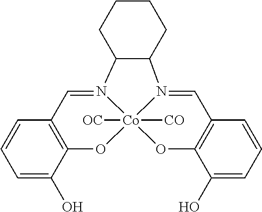

Catalyst XVI with a structure shown in the following is selected.

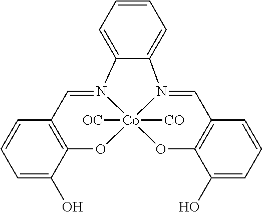

Catalyst XVII with a structure shown in the following is selected.

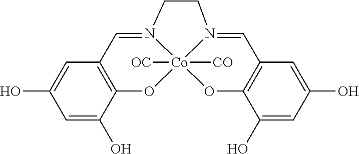

Catalyst XVIII with a structure shown in the following is selected.

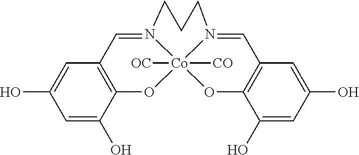

Catalyst XIX with a structure shown in the following is selected.

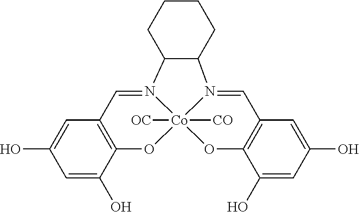

Catalyst XX with a structure shown in the following is selected.

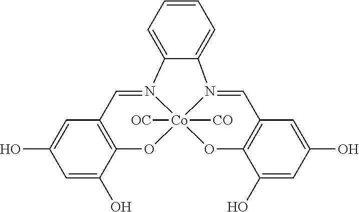

Table 4 Summary of reaction results

| | | EO Conversion | Selectivity of |
|---|---|---|---|
| Example | Catalyst | Rate (%) | 3-HMP (%) |
| 56 | II | 80.3 | 94.7 |
| 57 | III | 71.1 | 87.1 |
| 58 | IV | 83.9 | 75.4 |
| 59 | V | 91.1 | 79.5 |
| 60 | VI | 66.4 | 80.9 |
| 61 | VII | 43.6 | 72.8 |
| 62 | VIII | 79.4 | 86.1 |
| 63 | IX | 56.1 | 91.6 |
| 64 | X | 60.3 | 87.6 |
| 65 | XI | 86.9 | 73.9 |
| 66 | XII | 93.5 | 47.2 |
| 67 | XIII | 59.5 | 86.4 |
| 68 | XIV | 81.3 | 68.1 |
| 69 | XV | 92.4 | 81.5 |
| 70 | XVI | 40.5 | 76.4 |
| 71 | XVII | 76.9 | 73.8 |
| 72 | XVIII | 63.1 | 91.8 |
| 73 | XIX | 40.3 | 56.4 |
| 74 | XX | 83.6 | 49.8 |

The following is on the catalytic reaction of 3HP and dihydrogen.

The preparation procedure of the copper-containing mixed metal silicon oxide catalyst is described as follows.

A mixture of Cu(NO₃)₂.3H₂O and M′(NO₃)ₘ.n H₂O was dissolved in distilled water, and to it was added a silica sol solution when stirring. The mixture was allowed to heat to 60-95° C., and then to it was added a Na₂CO₃ solution. The solid precipitate was formed which reached to a maximum amount by aging for 4-8 h. The solid precipitate was collected by filtration, and washed thoroughly with deionized water till the Na⁺ and other metal ions was reduced in a maximum amount. The solid precipitate was dried at 120° C. for 24 h, and then calcined at 350-600° C. for 4-10 h to give a copper-containing mixed metal silicon oxide catalyst. On the basis of the original amount of reactants used as well as the amount of metal ions washed away, the formulas of the catalysts obtained are Zn_{0.1}Cu_{2.1}SiO_{3.3}, Mn_{0.15}Cu_{1.8}SiO_{3.5}, La_{0.3}Cu_{2.2}SiO_{3.6}, Zn_{0.1}Mn_{0.1}Cu_{1.7}SiO_{3.4}, Zn_{0.1}La_{0.2}Cu_{2.4}SiO_{3.5}, and Mn_{0.15}La_{0.2}Cu_{2.2}SiO_{3.6}, respectively.

The H₂-reduction activation of the copper-containing mixed metal silicon oxide catalyst is described as follows.

The catalyst was granulated into 40-70 meshes prior to use. 5 g catalyst was filled in the middle of a reaction tube, where the front and the end part of the tube were filled with the inert quartz sand or a ceramic circle. This filling should be compact to ensure a smooth and stable passing of the gas and the liquid species under a setting pressure during the reaction. To this catalyst-filled tube was pressurized with the reducing gas, H₂ (5% vol)/N₂, and the tube was subject to heat treatment. The first process was conducted by heating the tube from room temperature to 50° C. at a rate of 0.833° C./min. This step needed ca. 30 min. The second process was the further heating from 50° C. to 300° C. at a rate of 0.625° C./min. This step required ca. 400 min. At this temperature, the reducing gas was introduced, and this step treatment lasted for 550 min. After this, under the input of the forming reducing gas, the temperature was decreased to a settled temperature within 60 min for further use.

The H₂-hydrogenation process of 3-HP by using the forming activated copper-containing mixed metal silicon oxide catalyst is described as follows.

The forming reducing gas was switched to H₂ at the above settled temperature. The H₂ flow rate was set to 30-48 mL/min with the pressure in the range of 40-68 atm. A solution containing 3HP and an organic solvent with a volume ratio of 1:10 was pumped into the tube by using a metering pump at the flow rate of 0.02-0.10 mL/min. In the proceeding of the reaction, the reaction aliquot was picked up for analysis at the regular intervals. The results obtained are recorded in the table.

### Examples 75 to 111

These example results are obtained by selecting the catalyst Mn_{0.15}Cu_{1.8}SiO_{3.5}.

In order to fully evaluate the reactivity of the catalyst Mn_{0.15}Cu_{1.8}SiO_{3.5}, experiments were carried out by adopting varied conditions. Firstly, reactions were carried out by using 3-HMP as a reactant in hexane as a solvent at the H₂ pressure of 60 atm, where the flow rate of H₂ was set by 34 mL/min and that of 3-HMP by 0.06 mL/min and meanwhile the reaction temperature was settled at 140, 145, 150, 155, 160, 165, 170, 180, 190 and 200° C., respectively. The results obtained are recorded in Examples 75 to 84 in Table 5. Secondly, under the forming conditions, the reaction temperature was kept at 160° C. while the H₂ pressure was settled by varying from 40, 45, 50, 55, 58, 62, 64, 66, and finally to 68 atm. The results produced are recorded in Examples 85 to 93 in Table 5. Thirdly, by means of the reaction conditions applied in the first cases, the reaction temperature was kept at 160° C. while the H₂ flow rate was settled by 30, 32, 36, 38, 42, 45, and finally 48 mL/min. The results yielded are recorded in Examples 94 to 100 in Table 5. Fourthly, by means of the reaction conditions applied in the first cases, the reaction temperature was kept at 160° C. while the 3-HMP flow rate was settled by 0.02, 0.03, 0.04, 0.05, 0.08, and finally 0.10 mL/min. The results obtained are recorded in Examples 101 to 106 in Table 5. Fifthly, under the reaction conditions applied in the first cases, the reaction temperature was kept at 160° C. Meanwhile, the solvents such as pentane, benzene, methylbenzene, methanol and tetrahydrofuran were used instead of hexane. The results generated are recorded in Examples 107 to 111 in Table 5.

Table 5 Summary of the reaction results

| | | | | H₂ Flow | 3-HMP | 3-HMP | Selectivity |
|---|---|---|---|---|---|---|---|
| | | Temperature | Pressure | Rate | Flow Rate | Conversion | of 1,3-PDO |
| Example | Solvent | (° C.) | (atm) | (mL/min) | (mL/min) | Rate (%) | (%) |
| 75 | Hexane | 140 | 60 | 34 | 0.06 | 36.1 | 56.2 |
| 76 | Hexane | 145 | 60 | 34 | 0.06 | 40.6 | 61.5 |
| 77 | Hexane | 150 | 60 | 34 | 0.06 | 56.2 | 50.2 |
| 78 | Hexane | 155 | 60 | 34 | 0.06 | 78.5 | 69.5 |
| 79 | Hexane | 160 | 60 | 34 | 0.06 | 95.6 | 81.3 |
| 80 | Hexane | 165 | 60 | 34 | 0.06 | 96.8 | 68.1 |
| 81 | Hexane | 170 | 60 | 34 | 0.06 | 97.2 | 60.4 |
| 82 | Hexane | 180 | 60 | 34 | 0.06 | 98.2 | 50.3 |
| 83 | Hexane | 190 | 60 | 34 | 0.06 | 99.1 | 40.2 |
| 84 | Hexane | 200 | 60 | 34 | 0.06 | 99.3 | 30.1 |
| 85 | Hexane | 160 | 40 | 34 | 0.06 | 23.1 | 10.5 |
| 86 | Hexane | 160 | 45 | 34 | 0.06 | 30.1 | 20.6 |
| 87 | Hexane | 160 | 50 | 34 | 0.06 | 42.3 | 67.1 |
| 88 | Hexane | 160 | 55 | 34 | 0.06 | 65.8 | 72.8 |
| 89 | Hexane | 160 | 58 | 34 | 0.06 | 92.1 | 80.3 |
| 90 | Hexane | 160 | 62 | 34 | 0.06 | 93.2 | 83.1 |
| 91 | Hexane | 160 | 64 | 34 | 0.06 | 94.0 | 85.4 |
| 92 | Hexane | 160 | 66 | 34 | 0.06 | 93.8 | 79.8 |
| 93 | Hexane | 160 | 68 | 34 | 0.06 | 96.5 | 76.9 |
| 94 | Hexane | 160 | 60 | 30 | 0.06 | 86.5 | 40.6 |
| 95 | Hexane | 160 | 60 | 32 | 0.06 | 83.9 | 80.4 |
| 96 | Hexane | 160 | 60 | 36 | 0.06 | 90.5 | 82.6 |
| 97 | Hexane | 160 | 60 | 38 | 0.06 | 87.6 | 62.8 |
| 98 | Hexane | 160 | 60 | 42 | 0.06 | 92.6 | 54.3 |
| 99 | Hexane | 160 | 60 | 45 | 0.06 | 82.6 | 50.9 |
| 100 | Hexane | 160 | 60 | 48 | 0.06 | 86.7 | 49.7 |
| 101 | Hexane | 160 | 60 | 34 | 0.02 | 43.6 | 53.2 |
| 102 | Hexane | 160 | 60 | 34 | 0.03 | 50.1 | 57.8 |
| 103 | Hexane | 160 | 60 | 34 | 0.04 | 60.4 | 60.2 |
| 104 | Hexane | 160 | 60 | 34 | 0.05 | 71.8 | 85.1 |
| 105 | Hexane | 160 | 60 | 34 | 0.08 | 86.2 | 80.3 |
| 106 | Hexane | 160 | 60 | 34 | 0.10 | 76.3 | 81.6 |
| 107 | Pentane | 160 | 60 | 34 | 0.06 | 86.4 | 81.7 |
| 108 | Benzene | 160 | 60 | 34 | 0.06 | 67.8 | 65.4 |
| 109 | Methylbenzene | 160 | 60 | 34 | 0.06 | 80.9 | 76.5 |
| 110 | Methanol | 160 | 60 | 34 | 0.06 | 89.2 | 78.9 |
| 111 | Tetrahydrofuran | 160 | 60 | 34 | 0.06 | 78.7 | 82.4 |

### Examples 112 to 118

In this section, the experiments are designed to study the catalytic hydrogenation reactivity of the catalyst Mn_{0.15}Cu_{1.8}SiO_{3.5} on different 3HP. Reactions were carried out in hexane as a solvent at the temperature of 160° C., the H₂ pressure of 60 atm, the H₂ flow rate of 34 mL/min and the 3HP flow rate of 0.06 mL/min. The results are recorded in Examples 112 to 118 in Table 6.

Table 6 Summary of the reaction results

| | | 3HP | Selectivity of |
|---|---|---|---|
| | | Conversion | 1,3-PDO |
| Example | Selectivity of 3HP | Rate (%) | (%) |
| 112 | Ethyl 3-hydroxypropionate | 80.6 | 76.9 |
| 113 | N-propyl 3-hydroxypropionate | 75.9 | 83.1 |
| 114 | Isopropyl 3-hydroxypropionate | 62.9 | 66.8 |
| 115 | N-butyl 3-hydroxypropionate | 76.4 | 70.1 |
| 116 | Isobutyl 3-hydroxypropionate | 43.2 | 58.2 |
| 117 | Tert-butyl 3-hydroxypropionate | 30.1 | 46.2 |
| 118 | N-pentyl 3-hydroxypropionate | 56.2 | 43.1 |

### Examples 119 to 123

In this section, the experiments are designed to study the catalytic reactivity of the catalysts such as Zn_{0.1}Cu_{2.1}SiO_{3.3}, La_{0.3}Cu_{2.2}SiO_{3.6}, Zn_{0.1}Mn_{0.1}Cu_{1.7}SiO_{3.4}, Zn_{0.1}La_{0.2}Cu_{2.4}SiO_{3.5} and Mn_{0.15}La_{0.2}Cu_{2.2}SiO_{3.6} on the 3-HMP H₂-hydrogenation. Reactions were carried out in hexane as a solvent at the temperature of 160° C., the H₂ pressure of 60 atm, the H₂ flow rate of 34 mL/min and the 3-HMP flow rate of 0.06 mL/min. The results are recorded in Examples 119 to 123 of Table 7.

Table 7 Summary of the reaction results

| | | 3-HMP | Selectivity of |
|---|---|---|---|
| | | Conversion | 1,3-PDO |
| Example | Catalyst | Rate (%) | (%) |
| 119 | Zn_{0.1}Cu_{2.1}SiO_{3.3} | 52.1 | 62.0 |
| 120 | La_{0.3}Cu_{2.2}SiO_{3.6} | 23.4 | 13.5 |
| 121 | Zn_{0.1}Mn_{0.1}Cu_{1.7}SiO_{3.4} | 86.3 | 89.8 |
| 122 | Zn_{0.1}La_{0.2}Cu_{2.4}SiO_{3.5} | 41.6 | 25.9 |
| 123 | Mn_{0.15}La_{0.2}Cu_{2.2}SiO_{3.6} | 39.2 | 8.9 |

In the present invention, the catalytic reaction examples of EO, carbon monoxide and alcohol revealed the guaranty for the preparation of the 3HP by using the related catalyst invented. Also guarantied is the H₂-hydrogenation of the 3HP to prepare the 1,3-PDO target. In the first step catalytic reaction, the 3HP product can be separated from the product system for the use in the subsequent catalytic hydrogenation reaction. In view of the whole combined two-step reaction, if the 3HP product was not separated, the production of the 1,3-PDO would not be effective. Especially noted is that the 1,3-PDO is not able to produce directly from the catalytic reaction of EO, carbon monoxide and alcohol. Moreover, these results show that it is necessary to use different catalyst system for each reaction in the process of the two-step reactions above-mentioned. Therefore, the invented technique is different from the hydroformylation on the reaction system of EO, carbon monoxide, and H₂, by means of whether the one-step or two-step method. The key point herein is the introduction of the alcohol and then the procedures and the catalysts are remarkably differed.

The results of the examples in Table 6 show that the series of the hydrogenation reactions of the 3HP produced the 1,3-PDO and the alcohol byproduct. The results of the examples in Table 2 show that the alcohol byproduct can be recycled to the reaction of EO and carbon monoxide to produce the 3HP.

What is claimed is:

1. A method for preparing 1,3-propanediol (1,3-PDO) by coupling ethylene oxide (EO) with syngas, comprising:
S1: subjecting EO, carbon monoxide and an alcohol molecule to ring opening-carbonylation-esterification to prepare 3-hydroxypropionate (3HP);
S2: hydrogenating the obtained 3HP to produce 1,3-PDO;
wherein the two steps use different catalysts, and an alcohol molecule byproduct generated in the second step can be used for the first step reaction.

2. The method according to claim 1, wherein S1 specifically comprises:
a) subjecting EO, carbon monoxide and alcohol to reaction in an organic solvent in the presence of a catalyst and an additive under required reaction conditions as settled temperature and pressure and controlled time;
b) conducting phase separation of the species obtained from the reaction system after reaction is completed, where an aqueous phase, an organic phase and a precipitate phase are formed and the 3HP product is maximally retained in the organic phase;
c) collecting the organic phase, from which the 3HP product is further subject to separation for use, or alternatively the 3HP-containing organic solution is for direct use in the subsequent reaction.

3. The method according to claim 1, wherein S2 specifically comprises:
d) subjecting the 3HP separated from 2-c) to reaction with dihydrogen in an organic solvent in the presence of a catalyst at settled temperature and pressure within controlled time;
or subjecting the 3HP-containing organic solution obtained in 2-c) to reaction with dihydrogen in the presence of a catalyst at settled temperature and pressure in controlled time; and
e) separating the species obtained from the reaction system, wherein 1,3-PDO, the alcohol byproduct and the other organic components are obtained respectively, and the alcohol byproduct can be recycled for 2-a).

4. The method according to claim 1, wherein in 2-a), the catalyst is a metal ligand complex, specifically, a N,O-ligand coordinated metal complex with the following structure character: 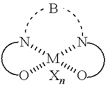
wherein, a metal M represents one of nickel, cobalt, ruthenium, rhodium, palladium, platinum, osmium, iridium, iron, copper and chromium, preferably one of cobalt, ruthenium, rhodium and iridium; a N,O ligand represents an organic group with N and O as the coordinating atom; N and O both have a σ-bond interaction with the metal M; B represents a bridging organic group connecting two N,O ligands by bonding to the nitrogen atom in the two ligands; X represents an anionic group or an atom or a neutral group; n represents a number of X; X and n fit to ensure the reliable oxidation state and coordination number of the central M after coordination by the N,O ligand; X is preferably one selected from H, CO, halogen, pseudohalogen, alkyl, alkoxyl, alkyl sulfydryl, aryl, benzyl, amino, hydroxyl and carboxylic group.

5. The method according to claim 1, wherein in 2-a), the additive is one selected from a basic metal oxide, a main group metal alkoxyl compound, a main group metal carboxyl compound, a metal carbonyl compound and a Lewis basic nitrogen-containing compound.

6. The method according to claim 1, wherein in 2-a), during the reaction of EO, carbon monoxide and the alcohol molecule in the organic solvent, the alcohol molecule is one selected from a C₁-C₂₀ alcohol, and preferably one selected from methanol, ethanol, propanol, butanol and pentanol; the organic solvent is one selected from alcohol, ether, saturated alkane and saturated aromatic hydrocarbons.

7. The method according to claim 1, wherein in 2-a), the reaction of EO, carbon monoxide and the alcohol molecule in the organic solvent is carried out at the temperature of 30-190° C. and the CO pressure of 1-150 atm for 0.1-200 h.

8. The method according to claim 1, wherein in 2-b), the reaction product system is separated by adding distilled water to generate the aqueous phase, adding an organic solvent to generate the organic phase, and adding a precipitating agent to generate the precipitate phase; the organic solvent is one selected from alcohol, ether, saturated alkane, saturated aromatic hydrocarbons, ester, C₅ and above long-chain olefin and C₄ and above long-chain alkyne; the precipitating agent is one selected from Bronsted acid, Bronsted base, Lewis acid, Lewis base, silica gel, molecular sieve, alumina, kaolin, hydrotalcite and ion exchange resin; the phase separation method is one selected from extraction, standing, centrifuge separation, filtration, distillation, column chromatography separation and vacuum extraction.

9. The method according to claim 1, wherein in 2-c), the 3HP product in the organic phase needs to be separated from the organic solvent by a method selected from distillation, rectification, column chromatography separation and vacuum extraction.

10. The method according to claim 1, wherein in the catalytic hydrogenation reaction of the 3HP in 3-d), the catalyst is a copper-containing mixed metal silicon oxide, and specifically, a copper-containing mixed metal silicon oxide having a general formula of M′ᵤCuᵥSi_{y}O_{z}, wherein u, v, y and z ensure to maintain a reliable M′ᵤCuᵥSi_{y}O_{z} formula molecule; M′ is one or two or three metals selected from zinc, manganese, barium, lanthanide series metal, cobalt, silver, gold, nickel and potassium.

11. The method according to claim 1, wherein in the catalytic hydrogenation reaction of the 3HP with dihydrogen in the organic solvent in 3-d), the organic solvent is one selected from ether, saturated alkane and saturated aromatic hydrocarbons; the reaction is carried out at the temperature of 80-400° C. and the H₂ pressure of 20-150 atm for 0.1-200 h.

12. The method according to claim 1, wherein after the catalytic hydrogenation reaction in 3-e), the product system is separated by a method selected from distillation, rectification, column chromatography separation and vacuum extraction; the alcohol byproduct obtained by the separation is recovered for the reaction according to claim 7.
